(19) United States
(12) Patent Application Publication
(10) Pub. No.: US 2012/0226187 A1
(43) Pub. Date: Sep. 6, 2012

(54) Vestibular Implant

(75) Inventors: Steven Bierer, Seattle, WA (US); Albert Fuchs, Seattle, WA (US); Leo Ling, Seattle, WA (US); Kaibao Nie, Bothell, WA (US); James Phillips, Seattle, WA (US); Jay Rubinstein, Seattle, WA (US)

(73) Assignee: University of Washington Center for Commercialization, Seattle, WA (US)

(21) Appl. No.: 13/375,196

(22) PCT Filed: May 28, 2010
(86) PCT No.: PCT/US10/36729

§ 371 Date: Apr. 27, 2012

Related U.S. Application Data
(60) Provisional application No. 61/182,526, filed on May 29, 2009.
(60) Provisional application No. 61/182,534, filed on May 29, 2009.

(51) Int. Cl. A61B 5/053; A61F 11/04; A61N 1/36

(52) U.S. Cl. 600/559; 607/57

(57) ABSTRACT

A vestibular implant comprising a stimulation unit to generate electric stimuli and deliver to electrode arrays is disclosed. The stimulation unit generates electric stimuli in response to a user input or continuously generates electrical stimuli. The electrode arrays comprise electrodes and are adapted for placement within a semicircular canal of an ear. The electric stimuli is delivered from the stimulation unit to the electrode so that the electrodes apply electric stimuli. A predetermined electric stimulus is applied to restore spontaneous vestibular activity during a Meniere's attack. A continuous, unmodulated electric stimulus is applied to suppress the symptoms of unilateral loss of vestibular function. Additionally, the electrodes record electrically evoked compound action potentials (eCAP). An appropriate location for the placement of the electrode array is determined based on the recorded eCAP.

### CROSS REFERENCE TO RELATED APPLICATIONS

This application claims the benefit of U.S. Provisional Application No. 61/182,534, filed May 29, 2009, and U.S. Provisional Application No. 61/182,526, filed May 29, 2009, each of which is incorporated by reference in its entirety.

### STATEMENT REGARDING FEDERALLY SPONSORED RESEARCH OR DEVELOPMENT

This invention was made with government support under HHS-N-260-2006-00005-C (NIDCD) awarded by the National Institutes of Health (NIH). The government has certain rights in this invention.

### BACKGROUND

This invention relates generally to vestibular implant devices and, more specifically, to the installation and use of a vestibular implant device capable of restoring unilateral loss of vestibular function and/or restoring spontaneous type activity to vestibular nerve during Meniere's attacks.

Vestibular disorders are very common and often debilitating. People of all ages and backgrounds are affected. Loss or disruption of normal vestibular function results in vertigo, loss of balance and orientation, and falls. Ten percent of patients with vestibular complaints are disabled by their vestibular loss. Vestibular disorders can be idiopathic. Drugs and environmental toxins can adversely affect the inner ear, as can viral infection and a host of genetic disorders.

The social cost of vestibular disorders is also significant. It is predicted that ninety five million Americans, or about 36% of the population, will visit their physician with a primary complaint of dizziness at least once in their lifetime. Society bears the burden of lost work and productivity and the cost of treatment for these individuals. This cost has been estimated to exceed $1 billion per year in the United States. Additional studies show that 5.4 million Americans visit their physician with a balance complaint annually, placing balance disorders among the top 25 most common reasons that Americans seek health care. With an aging population, the incidence and cost of balance disorders will increase as balance disorders increase in prevalence with increasing age. By age 75, balance disorders are one of the most common reasons for seeking health care. The majority of individuals over the age of 70 report problems with dizziness and imbalance and ⅓ of individuals aged 65 to 75 reports that balance problems adversely affect their quality of life.

Thus, prevalence of these problems illustrate that it is a large and growing public health problem. For example, the prevalence of dizziness ranges from 1.8% in young adults to greater than 30% in older adults. 65% of persons 60 years of age and older have chronic dizziness. 23-30% of adults have had at least one episode of dizziness and 3.5% of adults experience a chronic recurrent episode greater than a one-year duration by age 65. Approximately 12.5 million Americans over the age of 65 have a dizziness or balance problem that significantly interferes with their lives.

Treatment of vestibular disorders remains primarily based on therapies developed decades ago. With the exception of vestibular rehabilitation, which has expanded greatly over the past 10 years, most drugs and surgical therapies have not changed for over 20 years. Vestibular disorders have been the subject of meetings, seminars, publications, and research grants. These activities have reflected and supported substantial increases in knowledge about the effects of various diseases on balance, and in particular, the effects of aging. Nonetheless, there have been no significant new therapies translated to clinical practice. For example, we still rely on destructive procedures to correct the irreversibly diseased inner ear. Unlike the auditory system, where there are now developing notions of factors governing vulnerability to the major causes of deafness, most of the common vestibular disorders, such as Meniere's disease and vestibular neuronitis, remain poorly understood, etiologically and clinically.

Vestibular disorders can produce paroxysmal attacks of debilitating whirling vertigo and nausea lasting for hours and occurring as frequently as three times a week. While this condition is usually treated medically, at least fifteen percent of patients progress to requiring some sort of surgical intervention including vestibular nerve section, labyrinthectomy, endolymphatic shunt, or intratympanic gentamycin instillation. These procedures put residual hearing at risk to a greater or lesser degree and treatment is typically tailored to the degree of hearing loss in the affected ear. With the exception of the, endolymphatic shunt procedure, which is typically well tolerated but has a significant long-term failure rate, all of these interventions attempt to ablate vestibular function. While ablation is preferable to ongoing uncontrolled Meniere's attacks, the resulting profound unilateral vestibulopathy produces other symptoms that may pose problems for the patient, particularly if Meniere's or another disease process involves the contralateral ear.

Accordingly, what is needed are improved devices and procedures that can more reliably halt Meniere's attacks and/or restore unilateral loss of vestibular function, with a low risk of hearing loss or further loss of vestibular function.

### SUMMARY

To determine an accurate placement of an electrode within a semicircular canal of an ear, the disclosed embodiments provide an apparatus and a method for measuring electrically evoked compound action potentials within a semicircular canal of an ear. In one embodiment, a vestibular implant is communicatively coupled to a one or more electrode arrays. The electrode arrays are adapted for placement within a semicircular canal of an ear such that the electrode array does not compress the membranous canal. The electrode arrays comprise one or more electrodes. In one embodiment, one or more electrodes measure the electrically evoked compound action potentials within a semicircular canal. In another embodiment, the measured compound action potentials are sent to a display system, configured to display the measured compound action potentials within a semicircular canal.

The disclosed embodiments of the invention provide an apparatus and a method for effectively measuring and displaying compound action potentials within a semicircular canal. Because a high measure of compound action potentials is correlated with a more effective eye movements and consequently higher vestibulo-ocular reflex (VOR), such measurements allow doctors to place an electrode within a semicircular canal at an effective location. For example, if an electrode is placed in a location with low to zero compound action potential, electric stimuli applied to that region will not produce a desired effect of stimulating eye movements. In such cases, a highly invasive surgery would have to be performed again, potentially damaging several functionalities of the ear. Thus, the disclosed embodiments of the invention permit a more precise and effective placement of electrode arrays within a semicircular canal.

To restore afferent activity within a semicircular canal of an ear, the disclosed embodiments provide an apparatus and a method for applying electric stimuli to one or more semicircular canals of an ear. In one embodiment, a vestibular implant is communicatively coupled to an electrode array comprising one or more electrodes. An electrode array is adapted for placement within a semicircular canal of an ear. In one embodiment, a stimulator unit housed in a vestibular implant generates electrical stimuli and delivers the stimuli to one or more communicatively coupled electrode arrays. In such an embodiment, the electrodes housed in the electrode array apply the delivered electric stimuli. In an embodiment, the stimulator generates an electric pulse train based on a predetermined frequency and amplitude modulation in response to a user input. In another embodiment, the stimulator generates a continuous, unmodulated electric pulse train to deliver to one or more electrode arrays.

Vestibular disorders are associated with a loss of afferent activity. For example, the vertigo symptom of Meniere's disease is caused by a loss of spontaneous afferent activity in an ear. Similarly, loss of balance and nausea associated with unilateral vestibulopathy is caused by loss of continuous afferent activity in an ear. The disclosed embodiments of the invention alleviate the symptoms caused by such as loss by providing electric stimuli to certain portions of the semicircular canal. In one embodiment, the electric stimulus mimics spontaneous afferent activity, thereby reducing the vertigo of a Meniere's attack. In another embodiment, the electric stimuli are continuous and unmodulated, thereby reduce the loss of balance associated with unilateral loss of vestibular function. Thus, the disclosed embodiments of the invention provide an apparatus and a method for applying electric stimuli to stop or reduce the vertigo symptoms associated with Meniere's disease and unilateral loss of vestibular function.

### BRIEF DESCRIPTION OF THE DRAWINGS

FIG. 1 is a high-level block diagram illustrating an embodiment of a vestibular implant device.

FIG. 2 is a diagram illustrating an embodiment of an external device to control the vestibular implant device.

FIG. 3 is an illustration of a graphical user interface to input vestibular stimulation data.

FIG. 4 is an illustration of a system to reduce artifacts in recorded electrically-evoked compound action potentials.

FIG. 5 is an illustration of an embodiment of a system used to provide stimulation data to a vestibular implant device.

FIG. 6 is an illustration of semicircular canals fenestrated near their respective amullae for the insertion of electrode arrays.

FIG. 7a is a diagram illustrating an embodiment of an electrode array.

FIG. 7b illustrates an embodiment of the trajectory of an electrode array within a semicircular canal once inserted into a canal fenestration.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Implant Device for Restoring Vestibular Function

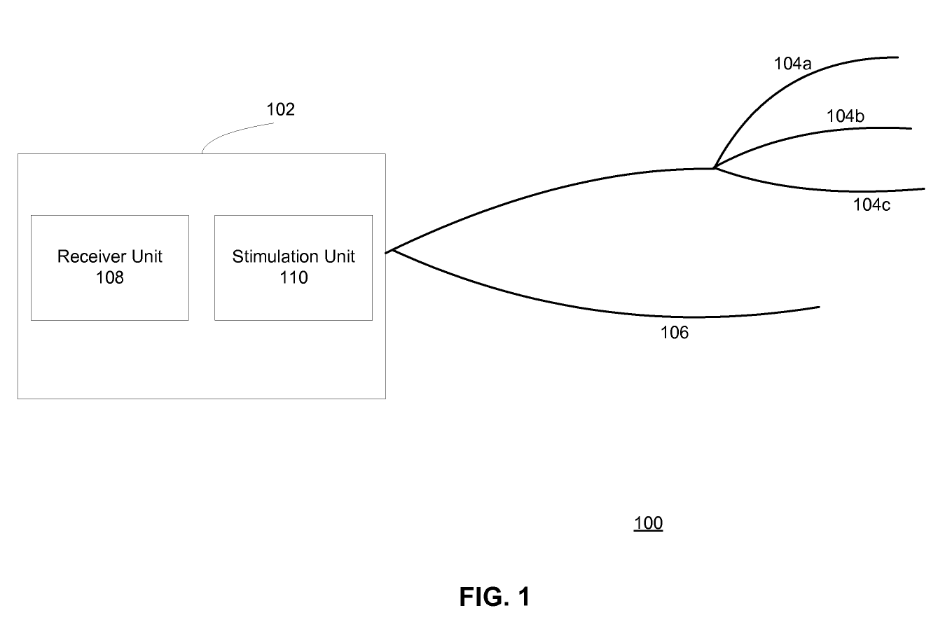

FIG. 1 illustrates an example apparatus 100 to stimulate portions of a vestibular canal according to an embodiment disclosed herein. The apparatus contains a receiver-stimulator 102, which is connectively coupled to electrode arrays 104 and a ball ground electrode 106. The receiver-stimulator 102 comprises a receiving unit 108 and a stimulation unit 110. In one embodiment, the receiver-stimulator 102 receives instructions from an external unit 200, described in greater detail below. Responsive to the instructions, the receiver-stimulator 102 outputs a stimulation signal to the electrodes within the electrode arrays 104. The electrodes apply the received electrical stimulation directly to the portion of the semicircular canal in contact with the electrodes. In an embodiment where the electrode arrays 104 are implanted in one or more semicircular canals of an ear, the apparatus 100 may be used to provide electrical stimulation to afferents near the semicircular canal as well as record vestibular electrically-evoked compound action potentials.

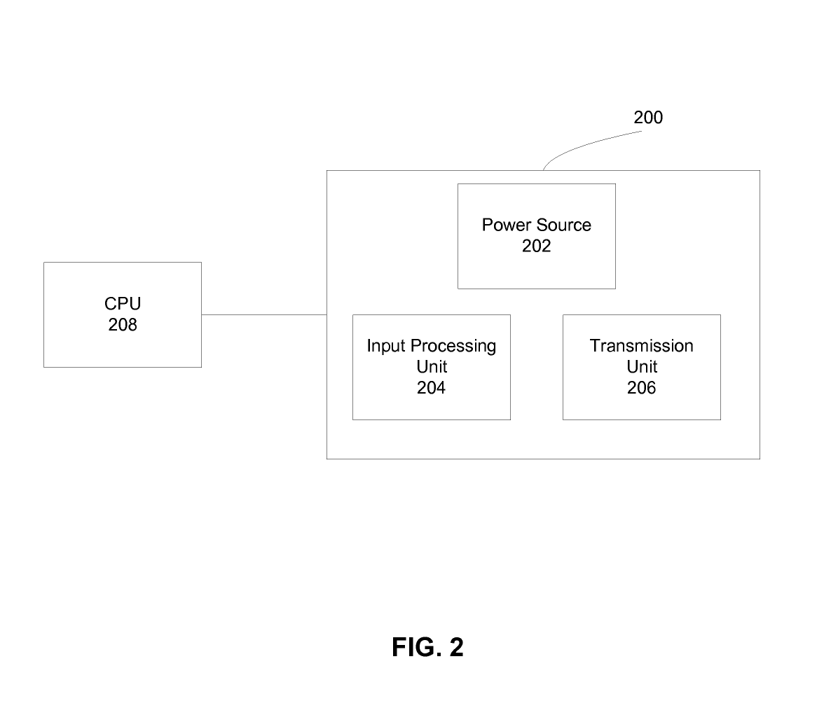

The receiver-stimulator 102 comprises an internal receiver unit 108 and a stimulator unit 110. The receiver unit 108 receives signals from an external unit 200. FIG. 2 illustrates an example of an external unit 200 according to an embodiment disclosed herein. In one embodiment, the external unit 200 comprises an input processing unit 204, a power source 202 and a transmission unit 206.

The input processing unit 204 receives instructions regarding an electrical stimulus to send to a receiver-stimulator 102 from an attached computer 208 or another input source. In another embodiment, the signal to transmit to a receiver-stimulator 102 is predefined within the input processing unit. For example, the instructions to generate particular electric stimuli may be hard-coded within the input processing unit 204. In one embodiment, the input signal received from a computer 208 may instruct the input processing unit 204 to transmit a monopolar or biopolar pulse. In another embodiment, the input signal received from a computer 208 is an instruction, instructing the stimulator-receiver unit 102 to generate an electrical stimulus responsive to the instructions. In such an embodiment, the input processing unit 204 transmits such instructions to the stimulator-receiver unit 102.

In another embodiment, the input processing unit 204 transmits the signal to the transmission unit 206, to transmit the electric pulse information to the internal receiver unit 108. In one embodiment, the external transmission unit 206 comprises an external coil and a magnet secured directly or indirectly to an external coil. The external coils are used to transmit power and stimulation data to the internal receiver-stimulator unit 102. In certain embodiments, external coil transmits electrical signals (i.e., power and stimulation data) to internal coil via a radio frequency (RF) link, discussed in greater detail below.

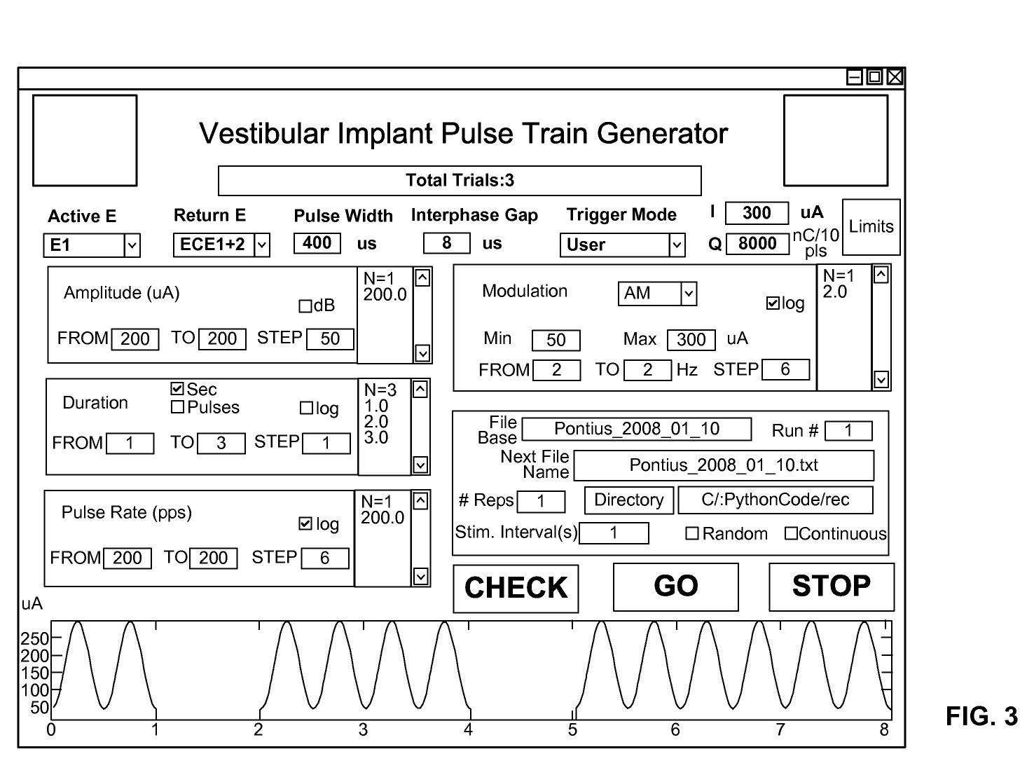

In one embodiment, a computer connected to the external unit is used to input stimulation data. FIG. 3 illustrates a graphical user interface for allowing users to deliver arbitrary stimuli to each of electrodes within an electrode array 104 under computer 208 control. Thus a user may user the graphical user interface 300 to control the implant device 100. For example, once the user changes an electric stimulus setting through the user interface, the computer 208 sends instructions to input processing unit 204. The input processing unit 204, in turn transmits the electric stimulus setting to the receiver unit 108 using a radiofrequency link to the implant.

In another embodiment, an external device 200 is not used. In such an embodiment, a pre-programmed processor within the internal device 102 is used to provide stimulation data to the receiver unit 108 or the stimulation unit 110.

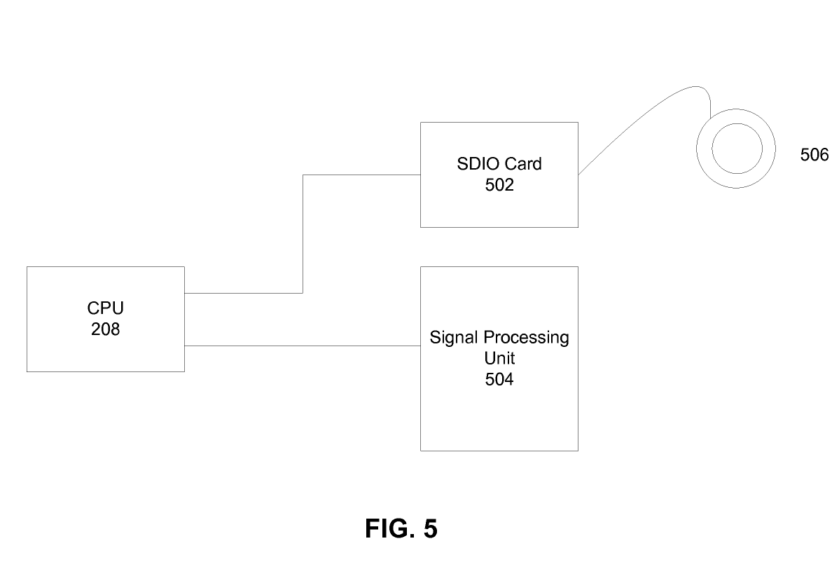

In another embodiment, a signal processor may be used as a stimulator. As illustrated in FIG. 5, a signal processing unit 504 uses a SDIO (Secure Digital Input/Output) card 502 to generate the radio frequency (RF) signal carrying stimulation parameters to an external coil 506. A computer 208 can program the signal processing unit 504 and download the pulse train generation codes to the signal processing unit 504 through a USB connection. The SDIO card 502 has a programmable FPGA for receiving control data and generating RF signals required by a specific communication protocol. The FPGA control code is downloaded from the PC by a JTAG link. Once both programs are downloaded, the signal processing unit 504 can be detached from the PC and servers as a stand-alone processor. Thus, the signal processing unit 504 can process any parameter change request immediately and can be also be potentially used to process rotational signals in real time.

In one embodiment, signal processing unit 504 stimulator codes can be used or modified to generate a desired pulse train on a specific electrode. To generate nystagmus from electrical stimulation of the vestibular end organ in a patient, a constant-rate and constant-amplitude pulse train may be used. For example, stimulation rate of 200 pps or 600 pps and a pulse width of 400 μs per phase on a single electrode (monopolar stimulation) may be used. Additionally, a specific FPGA file for the SDIO card 502 must be generated to produce the desired pulse train. Some unnecessary parts of the signal processing unit 504 stimulator program may need to be removed or changed to bypass the audio processing path in the original program. A new timer to control the package communication between the signal processing unit 504 and the SDIO card 502 may also need to be created. For example, a slider bar can added to the signal processing unit 504 control panel for varying the amplitude of the pulse train from 0 to 255 clinical levels.

Referring again to FIG. 1, the internal receiver unit 102 comprises an internal coil, and a magnet fixed relative to the internal coil. The magnets of the receiver unit facilitate the operational alignment of the external and internal coils, enabling internal coil to receive power and stimulation data from external coil. Internal coil is typically a wire antenna coil comprised of multiple turns of electrically insulated single-strand or multi-strand platinum or gold wire. The electrical insulation of internal coil is provided by a flexible silicone molding (not shown). The internal receiver-stimulation unit sends the received power and stimulation data to the communicatively coupled electrode arrays 104. Internal receiver unit and the simulator units are hermetically sealed within a biocompatible housing, shown as the receiver-stimulator unit 102.

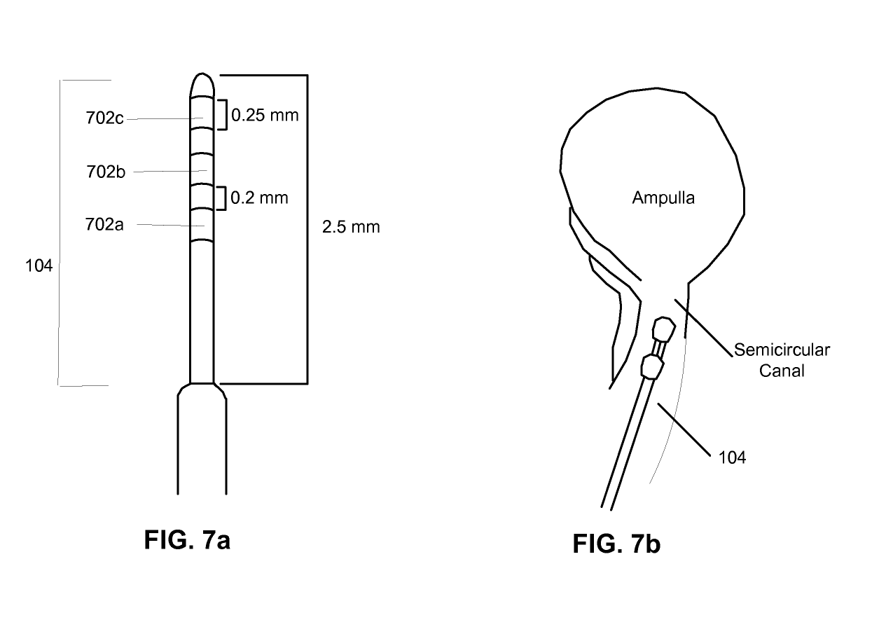

In one embodiment, the electrode arrays 104 have a proximal end connected to the receiver-stimulator unit 102 and a distal end that may be implanted in the semicircular canal of an ear. Referring to FIG. 1, the receiver-stimulator unit 102 is linked to a trifurcating array 104 of nine electrodes. Different embodiments use electrode arrays and electrode pads of varying sizes, each having varying characteristics and behavior once implanted in the semicircular canal. For example, when compared to a 1.7 mm array with 0.2 mm electrode pads an array of 2.5 mm array length and 0.25 mm electrode pads provides both device stability and improved impedance. An exemplary embodiment of an electrode array 104 comprising three electrode pads 702 is illustrated in FIG. 7a. The three electrodes 702 within the electrode array 104 are 0.2 mm in size. Additional, there is 0.2 mm of distance between each electrode pad 702. Finally, FIG. 7a illustrates that the electrode array is 2.5 mm in length. In one embodiment, the 0.25 mm electrode pads 702c have an impedance of approximately 10 kOhm in the operating room after placement within the labyrinth. Three electrode pads are included on each array to permit bipolar or monopolar stimulation as well as recordings of vestibular electrically-evoked compound action potentials. Small movement of the electrode array 104 causes the working array to become ineffective in generating nystagmus (discussed in greater detail below). Thus, in one embodiment, the length of the electrode array 104 tip is 3.4 mm, allowing for deeper insertion and a more robust and stable electrode array 104.

In one embodiment, an implanted canal may be activated using monopolar (MP) stimulation of the most proximal electrode of the three-electrode array, and recorded from the most distal electrode in the same canal. In another embodiment, separate return electrode may be used for stimulating and recording respectively when both the ball ground electrode and the receiver shell ground are available.

### Vestibular Implant for Measuring Compound Action Potentials

In one embodiment, the electrodes within the electrode array record electrically-evoked compound action potentials (eCAP) from the semicircular canals where the electrodes are inserted. The electrode measurements are recorded using a recording scope. In one embodiment, the recorded compound action potentials measured by an electrode and sent to a display unit configured to display the measured compound action potentials.

An evoked compound action potential generally occurs within micro seconds of the stimulation pulse. In one embodiment, a vestibular electrode array is driven by a processor and implant receiver. In an embodiment vestibular ECAP recordings are performed by software configured for stimulating one electrode, recording on an adjacent electrode, and then transmitting backward collected neural responses to a control computer.

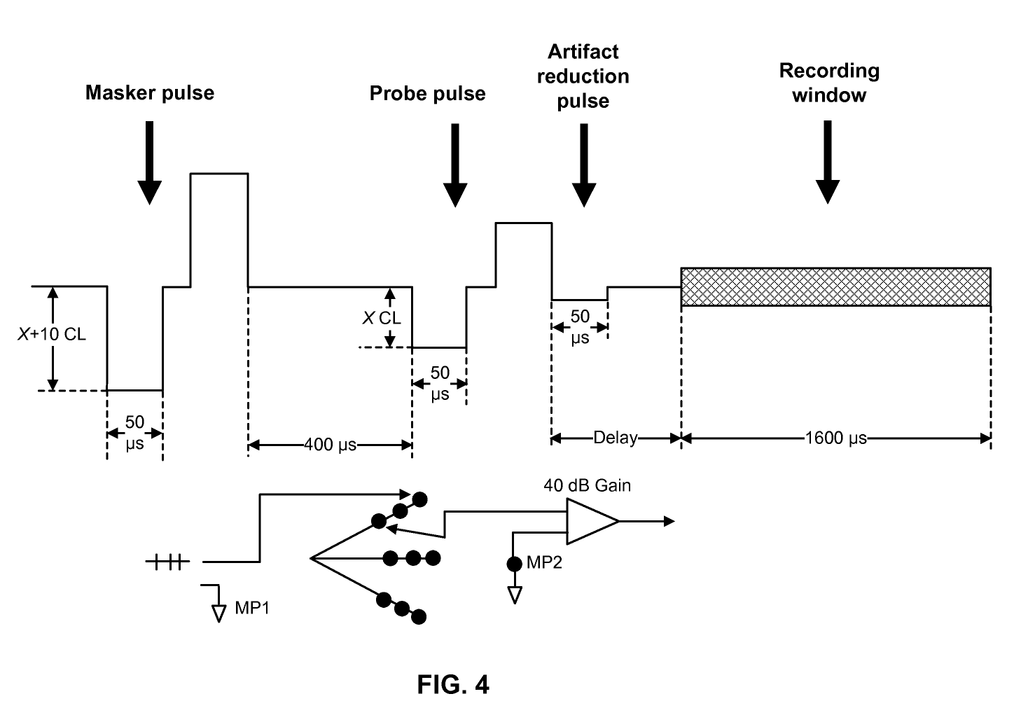

Neural responses usually overlap with the stimulating pulses, resulting in significant stimulation artifacts in ECAP recordings. It is an additional feature of the present apparatus to remove artifacts from the eCAP measurements recorded by one or more electrodes. One method to eliminate the artifact by characterizing the measurement response with the artifact and subtracting the measurement of the artifact alone. In another embodiment, a forward masking paradigm is adopted to minimize the artifacts associated with vestibular ECAP recordings. Some parameters were slightly varied across trials. FIG. 4 illustrates the experimental paradigm for artifact reduction in one embodiment of vestibular ECAP recordings. The forward masking paradigm consists of four phases of separate recordings. Phase A (Probe only): a biphasic probe pulse with varying current intensity is presented followed by a brief artifact reduction pulse. After the offset of the probe pulse; the recording window is opened for 1600 microseconds to record any neural activity produced by the probe pulse. Phase B (Masker+Probe): A masker pulse is presented 400 microseconds prior to the probe pulse. The masker pulse could eliminate the response to the probe pulse because the neural elements are in refractory period following presentation of the masker pulse. In this situation, the recording window records only the artifact resulting from the presentation of the probe pulse. Phase C (Masker only): the same masker pulse is presented to the stimulation electrode. Phase D (Switch-on): both, the probe and masker pulses are presented at a minimal level to produce artifacts caused by digital switching. Overall, subtracting recording A from artifacts in B, C and D (A-(B(C-D))) yields clean ECAP neural responses. In other embodiments, the artifact reduction pulse may be set to ‘off’ to minimize the delay of the recording window, allowing the better capture of the first negative peak.

To obtain the ECAP growth function, the amplitude of the probe pulse was progressively increased from a sub-threshold level up to the safest level by a step size of 10 clinical levels (CL). The relationship between the clinical level and the actual current in μA is defined as: I(μA)=17.5×100^{(x/255),} where x is in the range of 0-255 CLs.

Thus, an implant can generate a maximum current of 1750/μA when x is set to 255. However, the actual current limit of the implant may be subject to, for example 10V voltage compliance of the current source. For instance, the maximum current is around 1000 μA if the measured electrode impedance is 10 kΩ. As a result of the small-size electrode tip in the current electrode design, the electrode impedance of the vestibular prostheses in one embodiment is in the range of 25 kΩ to 30 kΩ, constraining the maximum current to be less than 400 μA.

In the embodiments where the amplitude of the masker pulse was 10 CLs higher than the probe pulse, the pulse width of each biphasic pulse was set to 50 μS for both the masker and the probe pulses. The interphasic gap was at 8 μS. If the artifact reduction pulse was present, it was also set to 50 μS in pulse width. Recordings were averaged over 50 presentations with a repetition rate of 80 Hz. The amplifier gain was always at 40 dB to avoid saturation.

### Method of Implanting the Implant Device for Effective Vestibular Stimulation

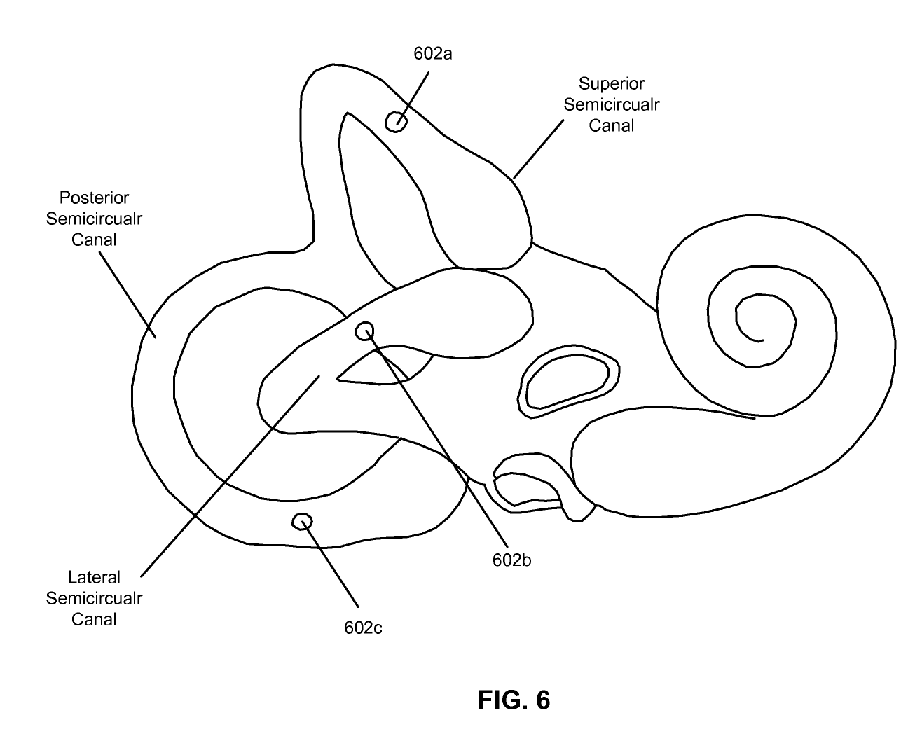

One method of implanting the disclosed apparatus is described below. In one embodiment, three electrode arrays are placed in the semicircular canals of an ear. As illustrated in FIG. 6, an electrode may be inserted in the semicircular canal at points 602 indicating an osseous opening for electrode arrays 104. Additionally, FIG. 7b illustrates that the electrode array 104 is inserted into the semicircular canal fenestration with a trajectory following the curvature of the canal. In the atraumatic surgery needed to place such devices without causing hearing loss, it is difficult to know precisely where an electrode lies relative to the target neuroepithelium. These recordings allow the surgeon to confirm that electrical activation of the target neurons is occurring and hence placement is adequate to evoke activation of the vestibular nerve.

In one embodiment of the method, the subject (or the patient) is prepped and draped in a sterile manner. A post-auricular incision is made with a #15 blade and carried down to the temporalis with a Bovie. A Palva flap is raised exposing the mastoid cortex. A sub-perisosteal pocket is created for the receiver-stimulator 102 posterosuperior to the external auditory canal. A simple mastoidectomy is performed exposing the incus and the target portions of the vestibular labyrinth. Each of the three semicircular canals can then be blue-lined with a 1 mm diamond burr, fenestrated with an angled pick as illustrated in FIG. 6 and electrodes inserted as shown in FIG. 7b using a jeweler's forceps after securing the receiver-stimulator 102 in its sub-perisosteal pocket. Care is taken to avoid suctioning perilymph from the canals after their fenestration and electrodes are inserted parallel to the trajectory of the canal so as to avoid penetrating the membranous labyrinth. The electrodes are implanted in one or more semicircular canals. Finally, fascia is used to seal the labyrinthotomy. Intraoperative electrophysiology is used to determine optimal electrode positioning in the canals as described below.

Precise electrode placement near the ampullae of the semicircular canals is important for robust ECAP responses. Electrode placement that is too shallow, or too deep, results in weak or absent vestibular responses. As described above, the vestibular afferents produce an electrically-evoked compound action potential (ECAP) that can be recorded from the implant using a software interface. Thus, in one embodiment, parts of the semicircular canals in contact with electrodes are stimulated and the resultant ECAP is recorded. The process is repeated until satisfactory ECAP results are obtained.

The benefit of the described surgical method is that it allows an exact placement of the implant device without damaging hearing or other functionalities of an ear. Although other single- or multi-unit recordings along the ascending vestibular pathway could also be used for studying the properties of vestibular neural firing by current excitation, such methods are invasive and are unsuitable for human recordings. The advantages of ECAP recording are: it can be conducted either when an animal or patient is in sedation during surgery or in awake status post surgery; it is a quick and safe test on whether current stimulation can produce any neural responses at the peripheral level. ECAPs from the auditory nerve have been successfully recorded in animals and in humans with cochlear implants. The morphology of auditory ECAPs provides an objective measure of the excitability of auditory fiber ensemble and it has been clinically proven useful for designing auditory prostheses and predicting subjective threshold for cochlear implant patients. The ECAP recoding is now a standard clinical tool available in most cochlear implant devices through backward telemetry. Stimulation artifact reduction methods, amplitude growth and latency of ECAPs have been extensively studied with cochlear implant patients.

### Restoring Spontaneous Activity During Meniere's Attacks

Meniere's disorder produces recurrent debilitating attacks of vertigo and nausea lasting for several hours. Its cause is believed to be recurring sudden loss of the normal spontaneous activity of the vestibular system in the affected ear. When the spontaneous activity recovers, the vertigo ceases. The disclosed system restores ‘spontaneous-like’ activity during the attacks through activation of the device described above, thus eliminating the source of the symptoms.

An apparatus to restore ‘spontaneous-like’ activity of vestibular system includes the implant device described above along with a triggering interface between eye-movement recording and vestibular implant activation systems. In one embodiment, the stimulation unit 110 is coupled to a user input. For example, a user may input a signal to activate the stimulation unit 110. Responsive to the user input, the stimulation unit 110 generates electric stimuli to deliver to one or more electrode arrays 104. The electric stimuli may comprise electric pulse trains of varying frequencies and amplitude modulations. For example, the electric stimuli are five short pulse trains on an electrode 104 with an extralyabyrinthine ground electrode 106 with parameters of 200 μA, 5 pulses, 200 pulses per second, 400 μs/phase, and 8 μs interphase gap. In other embodiments, other stimuli and combination of stimuli may be provided.

In another embodiment, the electric characteristics of stimuli to be generated are predetermined based on the effect electric stimuli has on a patient's eye movement and on the Meniere's attack symptoms. For example, eye-movements can be recorded responsive to one or more pulse trains to determine the effect of the pulse train on the eye movement. In one embodiment, the appropriate stimulus rate is set on the stimulation unit 110 somewhere between 0 and 300 pps. The effects of level and rate on the resulting eye-movements and any ensuing symptoms are determined. In the embodiment, based on recording of eye movements, different levels and frequencies of unmodulated pulse trains are mapped to a patient's stimulation unit 110 to allow different levels of vestibular activation to be set. For example, one level may be just below the threshold of eye movements, a second level can be set at the threshold eye movement level and a third level causes large and robust eye movements. These levels of stimulation allow a patient to set the implant device to a level appropriate to the severity of the vertigo attack. Furthermore, an appropriate level of stimulation prevents a level of stimulation so intense that it produces vertigo itself.

Once the electric stimuli are generated by the stimulation unit 110, the electric stimuli are delivered to one or more electrode arrays 104. The electrode array applies the delivered electric stimuli to the regions of the semicircular canal in contact with electrodes applying the stimuli. Activation of an electrode in the lateral semicircular canal by a biphasic pulse-train burst consistently evokes eye-movements in both vertical and horizontal directions. Experimentation has demonstrated both sustained vertical and horizontal eye movements in response to unmodulated pulse trains, as well as sinusoidal eye-movements in response to sinusoidally frequency-modulated pulse trains. The latter responses showed primarily horizontal eye-movements as desired. In one embodiment, the stimulation unit 110 is used to investigate current spread and search for combinations of stimulation parameters that produce the canal-specific eye-movements needed to produce a functional electrically-evoked vestibulo-ocular reflex (eVOR). VOR occurs when the vestibular system works with the visual system to keep objects in focus when the head is moving.

It is likely that more than just the lateral canal afferents are activated secondary to current spread. Since the effective amelioration of Meniere's attacks does not necessarily require canal-specific afferent activation, these subjects may attain significant benefit from the device while at the same time helping to define the stimulation parameters needed to treat a variety of other vestibulopathies. In one embodiment, an electrode is placed as far from the ampulla as possible so as to minimize potential loss of vestibular function. This increases spread of excitation which could be minimized by placing the electrodes closer to the ampulla. The more focused excitation comes at the cost of greater risk to residual vestibular function, a problem that would not be a concern in treating a patient who already has profound vestibular loss.

### System for Restoring Vestibular Functions in a Unilateral Vestibulopathy

Partial unilateral loss of vestibular function is a common clinical problem. Usually the central nervous system compensates for such unilateral loss over time and symptoms are self-limited. In a minority of cases, failure of central compensation results in permanent imbalance and unsteadiness that is resistant to existing therapeutic interventions.

To treat unilateral vestibulopathy, in one embodiment, the vestibular neurostimulation device 100 described above allows electrical activation of the vestibular periphery without further loss of hearing functions. In one embodiment, unmodulated ‘vestibular pacing’ is provided for unilateral vestibulopathy. For example, the stimulator unit 110 provides a steady amplitude electrical pulse to the electrodes 104. In another embodiment, once the threshold eCAP has been determined by the vestibular neurostimulation device 100, the stimulation unit 110 provides stimulation at a level slightly above or below the threshold eCAP levels. Thus, unmodulated pacing of the periphery via such a device enhances gain in a diseased labyrinth and suppresses the symptoms associate with unilateral loss of vestibular function.

### SUMMARY

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above disclosure.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all of the steps, operations, or processes described. Embodiments of the invention may also relate to an apparatus for performing the operations herein. Such a computer program may be stored in a tangible computer readable storage medium or any type of media suitable for storing electronic instructions, and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

What is claimed is:

1. An apparatus for measuring compound action potentials within a semicircular canal of an ear, the apparatus comprising:
a vestibular implant; and
a recording system configured to obtain electrically evoked compound action potentials within a semicircular canal of an ear, the compound action potentials measured by one or more electrode contacts within an electrode array adapted for placement within a semicircular canal.

2. The apparatus of claim 1, wherein the recording system measures compound action potentials and sends the measured data to a display system configured to display the measured compound action potentials.

3. The apparatus of claim 1, wherein the electrode array is 2.5 mm in length, the electrode contacts are 0.25 mm in diameter and have an impedance of 10 kOhm after placement in labyrinth of an ear.

4. A method for measuring compound action potentials within a semicircular canal of an ear the method comprising:
implanting a vestibular implant; and
measuring electrically evoked compound action potentials, the measure obtained by electrode contacts within an electrode array placed within the semicircular canal of the ear.

5. The method of claim 4, further comprising:
sending the electrically evoked compound action potential measurements to a display device, the measurements obtained by one or more electrode contacts within a semicircular canal and the display device configured to display the compound action potential measurements.

6. A vestibular implant apparatus, the apparatus comprising:
one or more electrode arrays adapted for placement within a semicircular canal of an ear, each array comprising one or more electrode contacts for applying electrical stimuli to tissue in contact with the electrode; and
a stimulator configured to generate electrical stimuli and deliver the electrical stimuli to a selected group of electrode contacts, such that at least one of the electrode contacts outputs an electrical current in the semicircular canal of an ear.

7. The apparatus of claim 6, wherein the stimulator is configured to generate one or more electric pulse-train bursts responsive to a user activation.

8. The apparatus of claim 6, wherein the stimulator generates one or more biphasic pulse-train bursts or sinusoidally frequency-modulated pulse trains responsive to user activation.

9. The apparatus of claim 6, wherein the stimulator generates a one or more electric pulse-train burst such that afferents near the electrode contacts are activated in response to the applied electrical stimuli.

10. The apparatus of claim 6, wherein the stimulator generates continuous, unmodulated electrical stimuli.

11. The apparatus of claim 6, wherein the stimulator generates a continuous, unmodulated electric pulse train such that afferents near the electrode contacts are continuously activated.

12. A method of restoring afferent activity within a semicircular canal of an ear, the method comprising:
placing an electrode array within the semicircular canal of the ear, the electrode array comprising one or more electrode contacts each configured to apply electrical stimuli to tissue near the electrode contact;
generating electrical stimuli using a stimulator unit; and
delivering electrical stimuli to one or more electrode contacts within a predetermined electrode array; and
applying the electrical stimuli through each electrode receiving electrical stimuli from the stimulator unit.

13. The method of claim 12, further comprising:
receiving instructions to generate a predetermined electrical stimuli, the instructions identifying the frequency and amplitude modulation of electrical pulse-trains to deliver to electrode contacts and apply to surrounding afferents.

14. The method of claim 12, further comprising:
generating electrical stimuli using the stimulator unit generates continuous, unmodulated electric pulse trains;
delivering each electric pulse train to one or more electrode contacts; and
applying the delivered electrical pulse trains using each one or more electrode contacts.
